(19) United States
(12) Patent Application Publication
(10) Pub. No.: US 2025/0312541 A1
(43) Pub. Date: Oct. 9, 2025

(54) A MEDICAMENT DELIVERY MEMBER ASSEMBLY

(71) Applicant: SHL Medical AG, Zug (CH)

(72) Inventor: Torbjörn Neby, Nacka Strand (SE)

(21) Appl. No.: 18/865,848

(22) PCT Filed: May 10, 2023
(86) PCT No.: PCT/EP2023/062350

§ 371 Date: Nov. 14, 2024

(30) Foreign Application Priority Data
May 24, 2022 (EP) 22175159.7

(51) Int. Cl. A61M 5/50; A61M 5/24; A61M 5/31

(52) U.S. Cl. CPC A61M 5/5086; A61M 2005/247; A61M 2005/312; A61M 2207/00

(57) ABSTRACT

The present disclosure provides a medicament delivery member assembly of a medicament delivery device. The medicament delivery member assembly includes a medicament delivery member extending along a longitudinal axis between a proximal end and a distal end, and a medicament delivery member holder comprising a body to which the medicament delivery member is attached. The body of the medicament delivery member holder comprises a distally extended wall extending in the distal direction of the medicament delivery member. The medicament delivery member assembly further includes an outer sleeve enclosing the medicament delivery member holder. The outer sleeve is axially movable relative to the medicament delivery member holder. The medicament delivery member assembly further includes a pierceable proximal seal operably sealing the proximal end of the medicament delivery member and a pierceable distal seal extending in a direction transverse to the longitudinal axis. The pierceable distal seal is attached to both the outer sleeve and the distally extended wall of the medicament delivery member holder, and the medicament delivery member is fully sealed by the outer sleeve, the proximal seal and the distal seal.

### CROSS-REFERENCE TO RELATED APPLICATIONS

The present application is a U.S. National Phase Application pursuant to 35 U.S.C. § 371 of International Application No. PCT/EP2023/062350 filed May 10, 2023, which claims priority to EP patent application Ser. No. 22/175,159.7 filed May 24, 2022. The entire disclosure contents of these applications are herewith incorporated by reference into the present application.

### TECHNICAL FIELD

The present disclosure generally relates to a medicament delivery member assembly, and particularly to a medicament delivery member assembly with a pierceable proximal seal and a pierceable distal seal.

### BACKGROUND

Medicament delivery devices such as pen type manual injectors or auto-injectors are generally known for the self-administration of a medicament by patients without formal medical training. For example, patients suffering from diabetes may require repeated injections of insulin, or patients may require regular injections of other types of medicaments such as a growth hormone.

Therefore, a clear operation guide for the patients is important. However, not every user reads a user guide or instructions on a package carefully every time before carrying out a medicament delivery operation, or sometimes unintentional access to the medicament delivery device might happen, e.g., accidentally detaching a cap of the medicament delivery device; or some end users need to perform multiple medicament delivery operations with multiple different medicament delivery devices and will be easily confused or mix up the medicament delivery device operation instructions between different medicament delivery devices.

Therefore, a structural design of the medicament delivery device that can make sure the users follow the operation instructions of the medicament delivery device is desirable. In particular, to make sure a medicament delivery member can only be unsealed when the user is ready for using the medicament delivery device.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

There is hence provided a medicament delivery member assembly of a medicament delivery device, the medicament delivery member assembly comprising: a medicament delivery member extending along a longitudinal axis between a proximal end and a distal end; a medicament delivery member holder comprising a body to which the medicament delivery member is attached, the body of the medicament delivery member holder comprises a distally extended wall extending in the distal direction of the medicament delivery member; an outer sleeve enclosing the medicament delivery member holder, the outer sleeve is axially movable relative to the medicament delivery member holder; a pierceable proximal seal operably sealing the proximal end of the medicament delivery member, and a pierceable distal seal extending in a direction transverse to the longitudinal axis. The pierceable distal seal is attached to both the outer sleeve and the distally extended wall of the medicament delivery member holder. The medicament delivery member is fully sealed by the outer sleeve, the proximal seal, and the distal seal. The medicament delivery member is sealed off by the outer sleeve, the proximal seal, and the distal seal. Therefore, the medicament delivery member is fully sealed from the ambient environment of the medicament delivery member assembly, so that the medicament delivery member can be keep in a sterilized state.

Preferably, according to another embodiment, a body of the medicament delivery member holder comprises the transverse wall perpendicular to the medicament delivery member; and the medicament delivery member is attached to the transverse wall.

Preferably, according to another embodiment, the outer sleeve comprises a proximal wall extending from a proximal end of the outer sleeve in the direction transverse to the longitudinal axis; and an aperture is in the proximal wall of the outer sleeve.

Preferably, according to another embodiment, the pierceable proximal seal extending from a proximal end of the outer sleeve in the direction transverse to the longitudinal axis; and the outer sleeve, the proximal seal and the distal seal define an enclosed chamber sealing the medicament delivery member inside the chamber.

Preferably, according to another embodiment, the pierceable proximal seal is placed on the aperture.

Alternatively, according to another embodiment, the medicament delivery member is wrapped by the pierceable proximal seal from the proximal end of the medicament delivery member to the medicament delivery member holder.

Preferably, according to another embodiment, the outer sleeve extends between a distal opening and the proximal wall. A diameter of the distal opening is greater than the aperture.

Preferably, according to another embodiment, the medicament delivery member holder comprises a fastener and the outer sleeve comprises a counter fastener engaged with the fastener of the medicament delivery member holder. The engagement formed by the fastener and the counter fastener is a groove and protrusion engagement, or a slot and protrusion engagement.

Preferably, according to another embodiment, the counter fastener extends from an inner surface of the outer sleeve. The fastener extends from the transverse wall of the medicament delivery member holder, or the fastener extends from an outer surface of the distally extended wall of the medicament delivery member holder.

Preferably, according to another embodiment, the distally directed wall of the medicament delivery member holder forms a sleeve body extending along the longitudinal axis and the pierceable distal seal is attached to both a distal end of the sleeve body of the medicament delivery member holder and a distal end of the outer sleeve in the direction transverse to the longitudinal axis.

Preferably, according to another embodiment, the distal seal comprises a proximally directed surface attached to both the outer sleeve and the distally extended wall of the medicament delivery member holder. The proximally directed surface of the distal seal is adhesive.

Preferably, according to another embodiment, the proximal seal is a membrane or a foil.

Preferably, according to another embodiment, the distal seal is a membrane or a foil.

Another aspect of the invention provides a sub-assembly of a medicament delivery device comprising the medicament delivery member assembly as mentioned above. The sub-assembly comprises a container carrier for accommodating a medicament container. The container carrier comprises a carrier body extending along the longitudinal axis between a distal end and the proximal end. The proximal end of the container carrier is aligned with the pierceable distal seal of the medicament delivery member assembly. The proximal end of the carrier body comprises a sharp protrusion; and the container carrier is movable relative to the medicament delivery member assembly along the longitudinal axis from a first position where the sharp protrusion is spaced apart from the pierceable distal seal, and a second position where the sharp protrusion is positioned through the pierceable distal seal.

Preferably, according to another embodiment, the sharp protrusion is a pair of sharp pins or a pair of blades.

Alternatively, according to another embodiment, the sharp protrusion is an annular blade extending around the proximal end of the carrier body.

Preferably, according to another embodiment, the sub-assembly comprises a housing extending along the longitudinal axis between a proximal end and a distal end.

Preferably, according to another embodiment, the container carrier is attached to the housing.

Alternatively, according to another embodiment, the medicament delivery member assembly is attached to the proximal end of the housing.

Preferably, according to another embodiment, a flexible arm extends from a wall of the housing; the flexible arm is radially flexible relative to the longitudinal axis. The flexible arm comprises a transverse ledge positioned between the proximal end of the container carrier and the pierceable distal seal.

Preferably, according to another embodiment, the sub-assembly comprises a medicament delivery member cover telescopically received in the proximal end of the housing; the medicament delivery member cover comprises a distally directed surface adjacent to a proximal end of the outer sleeve of the medicament delivery member assembly so that when the medicament delivery member cover moves in the distal direction relative to the housing, the medicament delivery member cover moves the medicament delivery member assembly towards the container carrier.

Preferably, according to another embodiment, the sub-assembly comprises a power source received within the housing. The power source is configured to apply force on the container holder in the proximal direction to the second position.

Preferably, according to another embodiment, the power source comprises a biasing member positioned between the distal end of the housing and the distal end of the container holder.

Preferably, according to another embodiment, the power source comprises a compression spring or a propellant gas canister.

Preferably, according to another embodiment, the sub-assembly comprises a plunger rod releasably and operably attached to the housing; the plunger rod extends along the longitudinal axis between a distal end and a proximal end; wherein the plunger rod comprises a distally directed surface. The biasing member is positioned between the distally directed surface of the plunger rod and the distal end of the housing. The proximal end of the plunger rod is configured to move into the medicament container within the container carrier from a distal end of the medicament container.

Preferably, according to another embodiment, the carrier body comprises a carrier fastener and the medicament delivery member holder of the medicament delivery member assembly comprises a medicament delivery member fastener attached to the carrier fastener when the container carrier is in the second position relative to the medicament delivery member assembly. The carrier fastener and the medicament delivery member fastener form a snap-fit engagement.

Preferably, according to another embodiment, the medicament delivery device comprises a multiple-chamber medicament container arranged within the carrier.

Preferably, according to another embodiment, the medicament delivery device is an auto-injector or a manual insulin pen.

Preferably, according to another embodiment, the medicament container is a medicament container without a medicament delivery member.

Preferably, according to another embodiment, the medicament container is a glass cartridge, a plastic cartridge, or a collapsible bag.

Preferably, according to another embodiment, the medicament delivery member is an injection needle, an infusion catheter, or a spray nozzle.

The medicament delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of drugs that could be included in the medicament delivery devices described herein include, but are not limited to, antibodies, proteins, fusion proteins, peptibodies, polypeptides, pegylated proteins, protein fragments, protein analogues, protein variants, protein precursors, and/or protein derivatives. Exemplary drugs that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the medicament delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Furthermore, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

### BRIEF DESCRIPTION OF THE DRAWINGS

Embodiments of the inventive concept will now be described, by way of example only, with reference to the accompanying drawings, in which:

FIG. 1 schematically shows a cross-section view of a medicament delivery member assembly of the invention.

FIG. 2 schematically shows a cross-section view of a container carrier of the invention.

FIGS. 3A-C schematically show one example of an operation sequence of a sub-assembly of the invention comprising the medicament delivery member assembly of FIG. 1 and the container carrier of FIG. 2.

FIGS. 4A-C schematically show another example of an operation sequence of a sub-assembly of the invention comprising the medicament delivery member assembly of FIG. 1 and the container carrier of FIG. 2.

### DETAILED DESCRIPTION

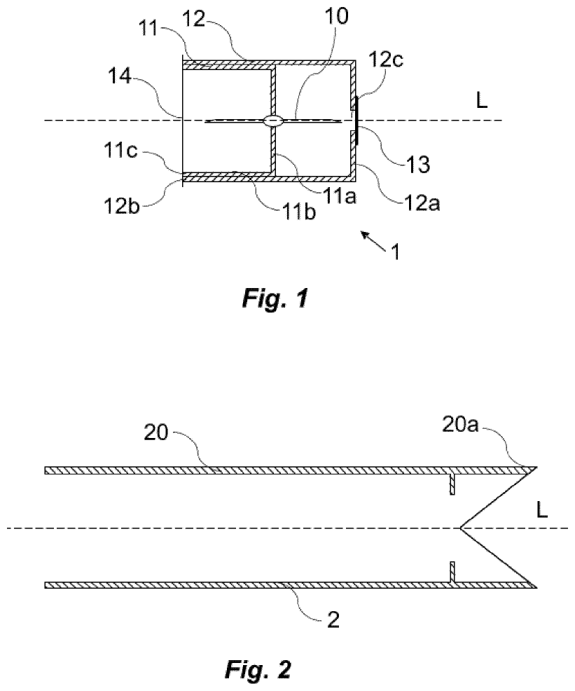

FIGS. 1-4C illustrate a medicament delivery member assembly of a medicament delivery device. The medicament delivery member assembly comprises a medicament delivery member 10 extending along a longitudinal axis L between a proximal end and a distal end. In a preferred example, the medicament delivery member is a needle, e.g., an injection needle. The medicament delivery member assembly comprises a medicament delivery member holder 11, an outer sleeve 12, a pierceable proximal seal 13 and a pierceable distal seal 14.

The medicament delivery member holder 11 comprises a body 11a to which the medicament delivery member 10 is attached. In one example, the body 11a comprises a transverse wall 11a perpendicular to the longitudinal axis L, as shown in FIG. 1. In this example, the medicament delivery member 10 is perpendicular to the transverse wall 11a and is extending through the transverse wall 11a along the longitudinal axis L. In this example, the transverse wall 11a comprises a central cavity and the medicament delivery member 10 is configured to be attached to a wall of the central cavity, e.g., by being glued to the wall of the central cavity or clamped by the wall of the central cavity. Alternatively, in another example, instead of the transverse wall, the body of the medicament delivery holder comprises a cone-shaped wall. In this example, a vertex of the cone-shaped wall is directed to the proximal end of the medicament delivery member. In this example, the vertex of the cone-shaped wall comprises a central cavity that the medicament delivery member is configured to be attached to a wall of the central cavity, e.g., by being glued to the wall of the central cavity or clamped by the wall of the central cavity.

The body 11a of the medicament delivery member holder 11 comprises a distally extended wall 11b extending in the distal direction of the medicament delivery member 10. In one example, the distally extended wall is two distally extended arms. In another example, the distally extended wall forms a sleeve body 11b extending along the longitudinal axis L.

The body 11a of the medicament delivery member holder 11 can be formed by either the transverse wall or the cone-shaped wall together with the distally extended wall 11b. Alternatively, the body 11a of the medicament delivery member holder can comprise other shapes of body sections dependent on the design.

The outer sleeve 12 extends in the direction of the longitudinal axis L between a proximal end and a distal end 12b. The outer sleeve 12 encloses the medicament delivery member holder 11. In one example, the medicament delivery member holder 11 is surrounded by the outer sleeve 12 as shown in FIG. 1. The outer sleeve 12 can be cylindrical or be a tube with a rectangular or a triangle cross-section (observed along the longitudinal axis). In one example, the outer sleeve comprises a distal opening at the distal end of the outer sleeve and a proximal opening at the proximal end of the outer sleeve. In one example, the distal opening and the proximal opening are circular. In one example where the distal opening and the proximal opening are circular, the distal opening has the same diameter as the proximal opening. Alternatively, the outer sleeve 12 comprises a proximal wall 12a extending from the proximal end of the outer sleeve 12 in a direction transverse to the longitudinal axis L. In this example, an aperture 12c is in the proximal wall 12a of the outer sleeve 12 as shown in FIG. 1. In this example, the outer sleeve 12 extends between the distal opening and the proximal wall 12a. In a preferred example, the aperture 12c and the distal opening of the outer sleeve are circular. In this example, a diameter of the distal opening is greater than the aperture.

The outer sleeve 12 is axially movable relative to the medicament delivery member holder 11. In one example, the medicament delivery member holder comprises a fastener and the outer sleeve comprises a counter fastener engaged with the fastener of the medicament delivery member hold. In this example, the engagement formed by the fastener and the counter fastener is a groove and protrusion engagement, or a slot and protrusion engagement. In one example, the counter fastener extends from an inner surface of the outer sleeve, and the fastener extends from an outer surface of the body of the medicament delivery member holder. Alternatively, the fastener extends from an outer surface of the distally extended wall of the medicament delivery member holder. In another example, there is no fastener between the medicament delivery member holder and the outer sleeve in the direction transverse to the longitudinal axis L. Therefore, the outer sleeve can slide along the medicament delivery member holder in the direction of the longitudinal axis L.

The pierceable proximal seal 13 operably seals the proximal end of the medicament delivery member 10. In one example, the pierceable proximal seal 13 extends from the proximal end of the outer sleeve 12 in the direction transverse to the longitudinal axis L. In one example where the outer sleeve comprises the proximal wall 11a and the aperture 12c, the proximal seal 13 is placed on the aperture 12c, as shown in FIG. 1.

Alternatively, the pierceable proximal seal is a rubber sheath. In this example, a portion of the medicament delivery member extending between the proximal end of the medicament delivery member and the medicament delivery member holder is wrapped by the pierceable proximal seal.

The pierceable distal seal 14 extends in the direction transverse to the longitudinal axis L. The pierceable distal seal 14 is attached to both the outer sleeve 12 and the distally extended wall 11b of the medicament delivery member holder 11, as shown in FIG. 1. Therefore, the medicament delivery member 10 is fully sealed by the outer sleeve 12, the proximal seal 13 and the distal seal 14.

In one example where the outer sleeve 12 comprises the proximal wall 12a and the aperture 12c, the outer sleeve 12, the proximal seal 13, and the distal seal 14 define an enclosed chamber sealing the medicament delivery member 10 inside the chamber, as shown in FIG. 1. In one example where the distally directed wall 11b of the medicament delivery member holder 11 forms the sleeve body 11b, the pierceable distal seal 14 is attached to both a distal end 11c of the sleeve body of the medicament delivery member holder 11 and a distal end 12b of the outer sleeve 12 in the direction transverse to the longitudinal axis L, as shown in FIG. 1.

In one example, the distal seal comprises a proximally directed surface attached to both the medicament delivery member holder 11 and the outer sleeve 12. The proximally directed surface of the distal seal is adhesive. In a preferred example, the proximally directed surface of the distal seal is attached to both the distal end of the medicament delivery member holder and the distal end of the outer sleeve. Therefore, the outer sleeve 12 cannot axially move relative to the medicament delivery member holder 11 unless the distal seal is ruptured or torn, e.g., so that the distal seal is split between the medicament delivery member holder and the outer sleeve. Therefore, the distal seal can no longer attach to both the medicament delivery member holder 11 and the outer sleeve 12. As mentioned above, the medicament delivery member 10 is fully sealed by the outer sleeve 12, the proximal seal 13 and the distal seal 14; therefore, only when the outer sleeve 12 is able to move relative to the medicament delivery member holder 11, the relative axial movement between the outer sleeve 12 and the medicament delivery member holder 11 can cause the proximal seal 13 to move towards the medicament delivery member 10. Thus, the medicament delivery member 10 can pierce the proximal seal 13. In one example, one or both of the proximal seal and the distal seal are membranes or foil.

In a preferred example, the proximal end of the outer sleeve 12 is configured to be pressed on the medicament delivery site during the use of a medicament delivery device comprising the medicament delivery member assembly. For example, the outer sleeve can be attached to a proximal end of the medicament delivery device.

A sub-assembly of the medicament delivery device is provided. The sub-assembly comprises the medicament delivery member assembly as mentioned above. The sub-assembly further comprises a container carrier 2; 2′ for accommodating a medicament container M, as shown in FIGS. 2-4C. The container carrier 2; 2′ comprises a carrier body 20 extending along the longitudinal axis L between a distal end and the proximal end. In a preferred example, the carrier body 20 is cylindrical, as the medicament containers comprise a cylindrical body. Alternatively, the carrier body can be any other suitable shape depending on the shape of the medicament container or the design of the medicament delivery device. In a preferred example, the container carrier comprises a conical proximal section pointing towards the distal end of the container carrier.

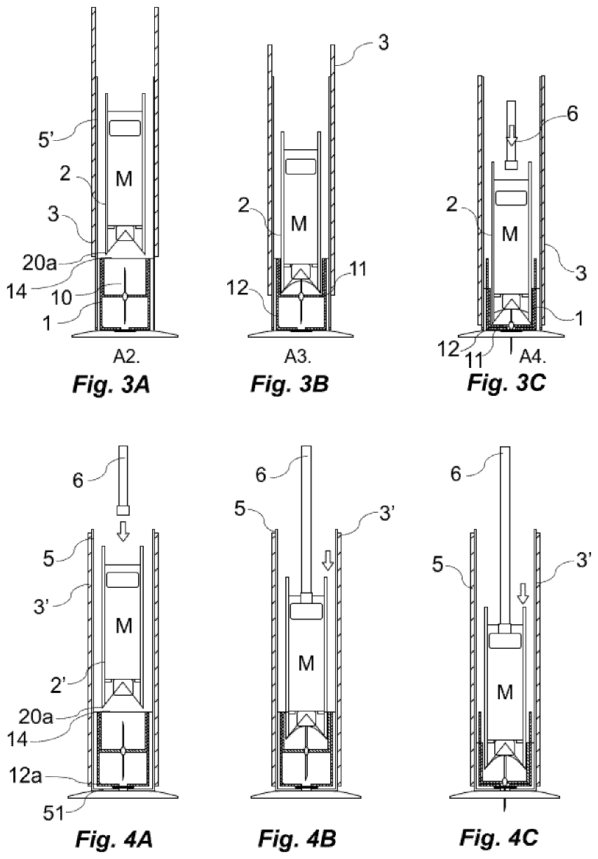

The proximal end of the container carrier 2; 2′ is aligned with the pierceable distal seal 14 of the medicament delivery member assembly 1, as shown in FIGS. 3A-4C. The proximal end of the carrier body 20 comprises a sharp protrusion 20a. In one example, the sharp protrusion 20a is a pair of sharp pins or a pair of blades. Alternatively, the sharp protrusion 20a is an annular blade extending around the proximal end of the carrier body.

The container carrier 2; 2′ is movable relative to the medicament delivery member assembly 1 along the longitudinal axis L from a first position where the sharp protrusion 20a is spaced apart from the pierceable distal seal 14, and a second position where the sharp protrusion 20a is positioned through the pierceable distal seal 14.

The sharp protrusion 20a of the container carrier 2; 2′ is configured to rupture or tear the pierceable distal seal 14, thereby enabling the relative axial movement between the outer sleeve 12 and the medicament delivery member hub 11 so that the proximal seal can be pierced by the medicament delivery member 10 once the outer sleeve 12 axially moves relative to the medicament delivery member hub 11. As the sharp protrusion 20a is arranged at the proximal end of the container carrier 2; 2′ that is configured to hold a medicament container M, the sharp protrusion 20a will only move towards the medicament delivery member assembly when a user plans to establish fluid communication between the medicament delivery member and the medicament container M, e.g., ready for carrying out a medicament delivery. Therefore, the medicament delivery member assembly can make sure that the proximal end of the medicament delivery member 10 will only be exposed to the user when the user is ready to carry out a medicament delivery operation.

In one example, the sub-assembly comprises a housing 3; 3′ extending along the longitudinal axis L between a proximal end and a distal end. The container carrier 2; 2′ is received within the housing 3; 3′. The housing can be cylindrical, or a tube with a rectangular, oval, or triangle cross-section when viewed along the longitudinal axis L, for example. The housing can be coated with texture for different targeted user groups to grip on. The housing can be arranged with an opening aligned with the container carrier so that the user can observe the contained medicament. Alternatively or additionally the housing can be transparent.

The medicament delivery member assembly 1 is at least partially received within the housing 3; 3′. The medicament delivery member assembly 1 can be attached to the proximal end of the housing or can be arranged to be movable relative to the housing at a predetermined distance. For example, an engagement between the medicament delivery member assembly and the housing can be a snap-fit engagement, so that the medicament delivery member assembly is immovable relative to the housing. Alternatively, the engagement between the medicament delivery member assembly and the housing can be a rib arranged within a groove so that the medicament delivery member assembly can axially move relative to the housing within the groove.

Similarly, the container carrier can be attached to the housing or can be arranged to be movable relative to the housing at a predetermined distance. For example, an engagement between the container carrier and the housing can be a snap-fit engagement, so that the container carrier is immovable relative to the housing. Alternatively, the engagement between the container carrier and the housing can be a rib arranged within a groove so that the container carrier can axially move relative to the housing within the groove.

It should be noted that the container carrier 2; 2′ is axially movable relative to the medicament delivery member assembly 1, therefore, if the container carrier is attached to the housing, the medicament delivery member assembly is configured to be axially movable relative to the housing by a predetermined distance. Similarly, if the medicament delivery member assembly is attached to the proximal end of the housing, the container carrier is configured to be axially movable relative to the housing by a predetermined distance. Alternatively, both the container carrier and the medicament delivery member assembly can be axially movable relative to the housing by a predetermined distance.

In one example where the medicament delivery member assembly 1 is attached to the proximal end of the housing, a flexible arm extends from a wall of the housing. In this example, the flexible arm is radially flexible relative to the longitudinal axis L and comprises a transverse ledge positioned between the proximal end of the container carrier and the pierceable distal seal. In this example, the sub-assembly preferably comprises a power source received within the housing. The power source is configured to deliver force to act on the distal end of the container holder. Therefore, when the force applied to the container holder deforms the flexible arm, the container holder can be moved towards the distal seal of the medicament delivery member assembly from the first position to the second position.

In one example, the power source comprises a biasing member positioned between the distal end of the housing and the distal end of the container holder. In a preferred example, the power source comprises a compression spring or a propellant gas canister. Alternatively, the power source comprises an electric motor. The power source can be only the biasing member or only the electric motor. Alternatively, the power source can be a combination of one or more biasing members with the electric motor.

In another example, the sub-assembly comprises a plunger rod 6 releasably and operably attached to the housing 3; 3′. The plunger rod 6 extends along the longitudinal axis L between a distal end and a proximal end. The plunger rod 6 comprises a distally directed surface. In this example, the power source is positioned between the distally directed surface of the plunger rod 6 and the distal end of the housing. In a preferred example, the power source comprises the biasing member in this example. In this example, the proximal end of the plunger rod is configured to move into the medicament container within the container carrier from a distal end of the medicament container. It should be noted that there are many different well-known ways of releasably attaching the plunger rod to the housing, and the way of releasably attaching the plunger rod to the housing is not a central part of the invention, thus, this description will not explain this in detail.

In one example, the sub-assembly comprises a medicament delivery member cover 5; 5′ telescopically received in the proximal end of the housing 3; 3′, as shown in FIGS. 3A-4C. The medicament delivery member cover 5; 5′ comprises a tubular proximal section for covering the medicament delivery member 10 after the use of the medicament delivery device. A proximal end of the tubular proximal section is configured to contact a medicament delivery site during the medicament delivery operation. Therefore, the user can move the medicament delivery member cover 5; 5′ towards the distal end of the housing during the medicament delivery operation. In a preferred example, the sub-assembly comprises a cover biasing member positioned between the medicament delivery member cover and the housing in the direction of the longitudinal axis L. Therefore, the medicament delivery member cover will be biased to cover the medicament delivery member until the user presses the proximal end of the tubular proximal section during the medicament delivery operation. It should be noted that, in one example where the outer shell 12 comprises the proximal wall 12a, the function of the medicament delivery member cover, namely, covering the medicament delivery member, can be replaced by the outer sleeve 12. In this example, the sub-assembly does not need a medicament delivery member cover.

In one example where the container carrier is attached to the housing, the medicament delivery member cover 5 comprises a distally directed surface 51 adjacent to a proximal end of the outer sleeve 12 of the medicament delivery member assembly 1, as shown in FIGS. 4A-4C, so that when the medicament delivery member cover moves in the distal direction relative to the housing, the medicament delivery member cover moves the medicament delivery member assembly towards the container carrier, thereby the container carrier is moved relative to the medicament delivery member from the first position to the second position. In one example where the outer sleeve comprises the proximal wall 12a, the distally directed surface 51 of the medicament delivery member cover 5 is adjacent to the proximal wall 12a. Alternatively, the sub-assembly comprises a cap covering the proximal end of the housing and the medicament delivery member assembly. In this example, the cap is configured to be pushed towards the distal end of the housing first before the cap can be removed. For example, the cap can be attached to the housing via a protrusion on the cap and a groove track in a wall of the housing.

It should be noted that the ways of moving the container carrier towards the medicament delivery member assembly and the ways of moving the medicament delivery member assembly towards the container carrier as mentioned above can be also used when both the medicament delivery member assembly and the container holder are axially movable relative to the housing as mentioned above. For example, FIGS. 3A-4C show that both the medicament delivery member assembly and the container holder are axially movable relative to the housing. FIGS. 3A-3C show one example where the distal seal is torn when the container holder moves towards the medicament delivery member assembly. Alternatively, FIGS. 4A-4C show one example where the distal seal is torn when the medicament delivery member assembly moves towards the container holder by the medicament delivery member cover 5.

In another example, the carrier body comprises a carrier fastener and the medicament delivery member holder of the medicament delivery member assembly comprises a medicament delivery member fastener attached to the carrier fastener when the container carrier is in the second position relative to the medicament delivery member assembly. In a preferred example, the carrier fastener and the medicament delivery member fastener form a snap-fit engagement. Therefore, when the container carrier moves to the second position, the medicament delivery member assembly is attached to the container carrier.

In one example where the medicament delivery member is a needle, the medicament delivery member assembly is configured to be used with a cartridge or a collapsible medicament container. In one example where the medicament delivery member assembly is used with a cartridge, the container carrier can be an optional component in this example. As a cartridge usually comprises a protruding proximal end, the protruding proximal end of the cartridge can push on the distal seal so that the distal seal is pierced by the needle. Once the distal seal is pierced by the needle, the proximal end of the cartridge can tear the distal seal along the ruptured part of the distal seal.

In a preferred example, the medicament delivery member assembly is preassembled in a medicament delivery device when the medicament delivery device is delivered to the user. As mentioned above, the relative movement between the container carrier and the medicament delivery member assembly is triggered when the user plans to use the medicament delivery device, e.g., is triggered by a movement of the medicament delivery member cover, is triggered by a movement of the cap removal, or is triggered by releasing the plunger rod. In one example where the medicament delivery member is the needle, during use of the medicament delivery device, the contain holder firstly tears the distal seal, as the user will press the proximal end of the outer sleeve against the medicament delivery site. Once the distal seal is torn, the outer sleeve will be moved towards the distal end of the housing, resulting in the needle piercing the proximal seal and the medicament delivery site. The continuous relative movement between the medicament delivery member assembly and the container holder before the medicament delivery member assembly attaches to the container holder causes the distal seal to be further pierced by a distal end of the needle, resulting in fluid communication between the needle and the contained medicament being established.

The inventive concept has mainly been described above with reference to a few examples. However, as is readily appreciated by a person skilled in the art, other embodiments than the ones disclosed above are equally possible within the scope of the inventive concept, as defined by the appended claims.

Some aspects of the invention are described in the clauses below.

1. A medicament delivery member assembly of a medicament delivery device, the medicament delivery member assembly comprising:
a medicament delivery member extending along a longitudinal axis between a proximal end and a distal end;
a medicament delivery member holder comprising a body to which the medicament delivery member is attached, wherein the body of the medicament delivery member holder comprises a distally extended wall extending in the distal direction of the medicament delivery member;
an outer sleeve enclosing the medicament delivery member holder; wherein the outer sleeve is axially movable relative to the medicament delivery member holder;
a pierceable proximal seal operably sealing the proximal end of the medicament delivery member; and
a pierceable distal seal extending in a direction transverse to the longitudinal axis, wherein the pierceable distal seal is attached to both the outer sleeve and the distally extended wall of the medicament delivery member holder; and
wherein the medicament delivery member is fully sealed by the outer sleeve, the proximal seal and the distal seal.

2. The medicament delivery member assembly according to clause 1, wherein a body of the medicament delivery member holder comprises the transverse wall perpendicular to the medicament delivery member; and wherein the medicament delivery member is attached to the transverse wall.

3. The medicament delivery member assembly according to clause 1 or 2, wherein the outer sleeve comprises a proximal wall extending from a proximal end of the outer sleeve in the direction transverse to the longitudinal axis; and wherein an aperture is in the proximal wall of the outer sleeve.

4. The medicament delivery member assembly according to any of the preceding clauses, wherein the pierceable proximal seal extends from a proximal end of the outer sleeve in the direction transverse to the longitudinal axis; and wherein the outer sleeve, the proximal seal and the distal seal define an enclosed chamber sealing the medicament delivery member inside the chamber.

5. The medicament delivery member assembly according to clause 4, wherein the pierceable proximal seal is placed on the aperture.

6. The medicament delivery member assembly according to clauses 1-3, wherein the medicament delivery member is wrapped by the pierceable proximal seal from the proximal end of the medicament delivery member to the medicament delivery member holder.

7. The medicament delivery member assembly according to any of clauses 2-5 and clause 6 when dependent on clause 3, wherein the outer sleeve extends between a distal opening and the proximal wall; and wherein a diameter of the distal opening is greater than the aperture.

8. The medicament delivery member assembly according to any of the preceding clauses, wherein the medicament delivery member holder comprises a fastener and the outer sleeve comprises a counter fastener engaged with the fastener of the medicament delivery member holder; and wherein in the engagement formed by the fastener and the counter fastener is a groove and protrusion engagement, or a slot and protrusion engagement.

9. The medicament delivery member assembly according to clause 8, wherein the counter fastener extends from an inner surface of the outer sleeve; and wherein the fastener extends from the transverse wall of the medicament delivery member holder, or wherein the fastener extends from an outer surface of the distally extended wall of the medicament delivery member holder.

10. The medicament delivery member assembly according to any of the preceding clauses, wherein the distally directed wall of the medicament delivery member holder forms a sleeve body extending along the longitudinal axis and wherein the pierceable distal seal is attached to both a distal end of the sleeve body of the medicament delivery member holder and a distal end of the outer sleeve in the direction transverse to the longitudinal axis.

11. The medicament delivery member assembly according to any of the preceding clauses, wherein the distal seal comprises a proximally directed surface attached to both the outer sleeve and the distally extended wall of the medicament delivery member holder; and wherein the proximally directed surface of the distal seal is adhesive.

12. The medicament delivery member assembly according to any of the preceding clauses, wherein the proximal seal and the distal seal are membranes.

13. A sub-assembly of a medicament delivery device comprising the medicament delivery member assembly according to any of the preceding clauses, the sub-assembly comprising:
a container carrier for accommodating a medicament container; wherein the container carrier comprises a carrier body extending along the longitudinal axis between a distal end and the proximal end;
wherein the proximal end of the container carrier is aligned with the pierceable distal seal of the medicament delivery member assembly; wherein the proximal end of the carrier body comprises a sharp protrusion; and wherein the container carrier is movable relative to the medicament delivery member assembly along the longitudinal axis from a first position where the sharp protrusion is spaced apart from the pierceable distal seal, and a second position where the sharp protrusion is positioned through the pierceable distal seal.

14. The sub-assembly according to clause 13, wherein the sharp protrusion is a pair of sharp pins, or a pair of blades.

15. The sub-assembly according to clause 13, wherein the sharp protrusion is an annular blade extending around the proximal end of the carrier body.

16. The sub-assembly according to any of clauses 13-15, comprising a housing extending along the longitudinal axis between a proximal end and a distal end.

17. The sub-assembly according to any of clauses 13-15, wherein the container carrier is attached within the housing.

18. The sub-assembly according to clause 17, wherein the medicament delivery member assembly is attached at the proximal end of the housing.

19. The sub-assembly according to clause 17 or 18, wherein a flexible arm extends from a wall of the housing; wherein the flexible arm is radially flexible relative to the longitudinal axis; and wherein the flexible arm comprises a transverse ledge positioned between the proximal end of the container carrier and the pierceable distal seal.

20. The sub-assembly according to any of clauses 17 or 18 or 19 when dependent on clause 17, comprising a medicament delivery member cover telescopically received in the proximal end of the housing;
wherein the medicament delivery member cover comprises a distally directed surface adjacent to a proximal end of the outer sleeve of the medicament delivery member assembly so that when the medicament delivery member cover moves in the distal direction relative to the housing, the medicament delivery member cover moves the medicament delivery member assembly towards the container carrier.

21. The sub-assembly according to clause 17 or clause 19 when dependent on clause 17, comprising a power source received within the housing; wherein the power source is configured to apply force on the container holder in the proximal direction to the second position.

22. The sub-assembly according to clause 21, wherein the power source comprises a biasing member positioned between the distal end of the housing and the distal end of the container holder.

23. The sub-assembly according to clause 22, wherein the power source comprises a compression spring or a propellant gas canister.

24. The sub-assembly according to clause 22 or 23, wherein the sub-assembly comprises a plunger rod releasably and operably attached to the housing; wherein the plunger rod extends along the longitudinal axis between a distal end and a proximal end; wherein the plunger rod comprises a distally directed surface; wherein the biasing member is positioned between the distally directed surface of the plunger rod and the distal end of the housing; and wherein the proximal end of the plunger rod is configured to move into the medicament container within the container carrier from a distal end of the medicament container.

25. The sub-assembly according to any one of clauses 13-24, wherein the carrier body comprises a carrier fastener and the medicament delivery member holder of the medicament delivery member assembly comprises a medicament delivery member fastener attached to the carrier fastener when the container carrier is in the second position relative to the medicament delivery member assembly; and wherein the carrier fastener and the medicament delivery member fastener forms a snap-fit engagement.

1-15 (canceled)

16. A medicament delivery member assembly of a medicament delivery device, the medicament delivery member assembly comprising:
a medicament delivery member extending along a longitudinal axis between a proximal end and a distal end;
a medicament delivery member holder comprising a body to which the medicament delivery member is attached, wherein the body of the medicament delivery member holder comprises a distally extended wall extending in the distal direction of the medicament delivery member;
an outer sleeve enclosing the medicament delivery member holder, wherein the outer sleeve is axially movable relative to the medicament delivery member holder;
a pierceable proximal seal operably sealing the proximal end of the medicament delivery member; and
a pierceable distal seal extending in a direction transverse to the longitudinal axis, wherein the pierceable distal seal is attached to both the outer sleeve and the distally extended wall of the medicament delivery member holder, and wherein the medicament delivery member is fully sealed by the outer sleeve, the proximal seal, and the distal seal.

17. The medicament delivery member assembly according to claim 16, wherein a body of the medicament delivery member holder comprises the transverse wall perpendicular to the medicament delivery member, and wherein the medicament delivery member is attached to the transverse wall.

18. The medicament delivery member assembly according to claim 16, wherein the outer sleeve comprises a proximal wall extending from a proximal end of the outer sleeve in the direction transverse to the longitudinal axis, and wherein an aperture is in the proximal wall of the outer sleeve.

19. The medicament delivery member assembly according to claim 16, wherein the pierceable proximal seal extends from a proximal end of the outer sleeve in the direction transverse to the longitudinal axis, and wherein the outer sleeve, the proximal seal and the distal seal define an enclosed chamber sealing the medicament delivery member inside the chamber.

20. The medicament delivery member assembly according to claim 19, wherein the pierceable proximal seal is placed on the aperture.

21. The medicament delivery member assembly according to claim 16, wherein the medicament delivery member is wrapped by the pierceable proximal seal from the proximal end of the medicament delivery member to the medicament delivery member holder.

22. The medicament delivery member assembly according to claim 18, wherein the outer sleeve extends between a distal opening and the proximal wall, and wherein a diameter of the distal opening is greater than the aperture.

23. The medicament delivery member assembly according to claim 16, wherein the medicament delivery member holder comprises a fastener and the outer sleeve comprises a counter fastener engaged with the fastener of the medicament delivery member holder, and wherein in the engagement formed by the fastener and the counter fastener is a groove and protrusion engagement, or a slot and protrusion engagement.

24. The medicament delivery member assembly according to claim 23, wherein the counter fastener extends from an inner surface of the outer sleeve, and wherein the fastener extends from the transverse wall of the medicament delivery member holder, or wherein the fastener extends from an outer surface of the distally extended wall of the medicament delivery member holder.

25. The medicament delivery member assembly according to claim 16, wherein the distally directed wall of the medicament delivery member holder forms a sleeve body extending along the longitudinal axis and wherein the pierceable distal seal is attached to both a distal end of the sleeve body of the medicament delivery member holder and a distal end of the outer sleeve in the direction transverse to the longitudinal axis.

26. The medicament delivery member assembly according to claim 16, wherein the distal seal comprises a proximally directed surface attached to both the outer sleeve and the distally extended wall of the medicament delivery member holder, and wherein the proximally directed surface of the distal seal is adhesive.

27. The medicament delivery member assembly according to claim 16, wherein the proximal seal and the distal seal are membranes.

28. A sub-assembly of a medicament delivery device comprising the medicament delivery member assembly according to any of the preceding claims, the sub-assembly comprising:
a container carrier for accommodating a medicament container, wherein the container carrier comprises a carrier body extending along the longitudinal axis between a distal end and the proximal end,
wherein the proximal end of the container carrier is aligned with the pierceable distal seal of the medicament delivery member assembly, wherein the proximal end of the carrier body comprises a sharp protrusion, and wherein the container carrier is movable relative to the medicament delivery member assembly along the longitudinal axis from a first position where the sharp protrusion is spaced apart from the pierceable distal seal, and a second position where the sharp protrusion is positioned through the pierceable distal seal.

29. The sub-assembly according to claim 28, comprising a housing extending along the longitudinal axis between a proximal end and a distal end.

30. The sub-assembly according to claim 29, comprising a power source received within the housing, wherein the power source is configured to apply force on the container holder in the proximal direction to the second position.
